(19) United States
(12) Patent Application Publication
(10) Pub. No.: US 2012/0309700 A1
(43) Pub. Date: Dec. 6, 2012

(54) DEVELOPMENT AND EVALUATION OF NOVEL CHINESE MATERIA MEDICA PREPARATIONS

(76) Inventors: Ching Song, Beijing (CN); Yuan Yi Xie, Beijing (CN); Lu Wang, Beijing (CN); Hao Yu, Beijing (CN); Shu Tsung Liao, Chicago, IL (US)

(21) Appl. No.: 13/499,864

(22) PCT Filed: Aug. 24, 2010
(86) PCT No.: PCT/CN10/76310

§ 371 Date: Aug. 23, 2012

(30) Foreign Application Priority Data
Aug. 27, 2009 (CN) 200910169622.6

(51) Int. Cl. A61K 31/704; A61K 31/58; C12Q 1/68; A61P 37/00; A61P 25/00; A61P 3/10; A61K 31/575; A61P 3/06

(52) U.S. Cl. 514/26; 514/182; 514/172; 435/6.13

(57) ABSTRACT

Nuclear receptor LXR is an important member of 49 nuclear receptors in human bodies and has irreplaceable regulatory effects on cholesterol and fat metabolisms. The regulation of the biological activity of LXR may have therapeutic effects to the conditions such as cardiovascular and cerebrovascular obstructions, non-insulin dependent hyperglycemia, immune function disorders, and central nerve functional degeneration. We have developed a group of novel Chinese materia medica preparations targeting nuclear receptor LXR by using a computer-simulated docking technology and a process for determining biological activities of human cells cultured ex vivo. The group of preparations are featured by simple ingredients, low costs, easy to prepare, etc. No significant adverse effects were found in initial acute toxicology analysis which was carried out using mice.

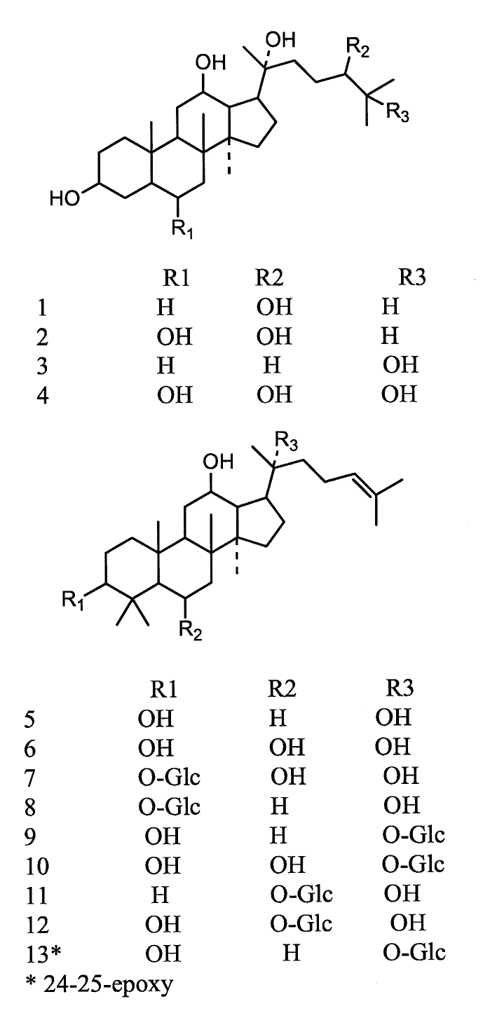

### TECHNICAL FIELD

Preparation and Evaluation of Chinese Materia Medica Preparations.

### BACKGROUND ART

Radix Ginseng is a commonly used type of traditional Chinese medicine drugs, and has remarkable effects of anti-fatigue, immunity-enhancing, strengthening Yang, etc., but its explanation of pharmacological actions on the molecular basis still needs to be determined. The components of Radix Ginseng are complicated, and different conventional processing techniques may produce different pharmacodynamic effects, which cause great difficulties for the identification and quantification of effective components. Currently, the pharmacological research on Radix Ginseng is divided into two aspects: (1) pharmacodynamic description and explanation; and (2) identification of effective components. Among them, the second aspect depends on the achievements in the first aspect. The research efforts to Radix Ginseng are mainly focused on experiments on living organisms, using living experimental animals or ex vivo cells to observe the changes of biological and chemical criteria, and the Radix Ginseng samples used are mostly extracted mixtures or singular pure compounds. The drawbacks in such experiments are mainly that indirect criteria are used for measurement and there is the lack of parallel tests carried out on positive and negative control drugs, therefore, it is very difficult to make comparison and generalization from different experimental data. As to the research efforts on effective components, currently they cover two aspects: the identification of the main components and the identification of the main metabolites. Dozens of different chemical structures have been analyzed from ginsenosides which are the unique main components of Radix Ginseng, while different species of Radix Ginseng and different processing techniques may generate new components and component ratios. The research efforts on the main metabolites have found out that the ginsenosides have very low absorptivity (<4%) in stomach and intestines, and moreover, the components of the main metabolites are singular (for example, compound K), which cannot give satisfactory explanation on many different pharmacodynamic effects by different Radix Ginseng preparations. Furthermore, the research efforts on the effective components with solid experimental data on, for instance, estrogen-like action, the effects to some cell membrane receptors and ion channels, etc., are very limited. Nevertheless, the compounds used in these research efforts are mostly in very high concentrations, making it difficult to achieve in human bodies.

According to the experimental data accumulated by now, it can be presumed that the genuine effective components contained in Radix Ginseng are yet to be determined. These components should have the following characteristics: (1) With high affinity to drug targets, since the main components (ginsenosides) of Radix Ginseng have very low absorptivity, therefore, the small amounts of absorbed substances are in very low concentrations and their pharmacological effects should be realized via high-affinity action sites. (2) Unstable chemical structures, due to the changes of processing methods, the pharmacological effects of Radix Ginseng are also changed accordingly. This implies that the effective components therein have poor stability. (3) Co-existed components of similar structures may have opposite pharmacological effects.

Based on the above-mentioned presumptions, we made investigation in using a computer-aided calculation method to identify effective components of Radix Ginseng. The calculation method calculated the interactions between the non-covalent bonds within macromolecules by applying Newtonian mechanics with the aid of PAULING atomic radius and the experimental ionization constant. At the moment, the structures of proteins, including large quantity of structural proteins and regulatory factors, have been generally resolved by X-ray diffraction crystal and high-resolution nuclear magnetic resonance; and the structural information is loaded into public databases. In addition, the structures of ginsenosides have been also resolved and published. These work bases provide a possibility in finding high-affinity binding sites with computer-aided calculation. It has been shown primarily by the computer-simulated docking results that parts of ginsenosides may have high-affinity binding to several nuclear receptors, thus activating or inhibiting the biological activities of these receptors, to produce opposite pharmacodynamic effects. These progresses not only have enriched our understandings of the effects of Radix Ginseng, but also provided new direction for the research on the physiological functions of these nuclear receptors.

### REFERENCE DOCUMENTS

1. Williams S et al. (2003) X-ray crystal structure of the liver X receptor beta ligand binding domain: regulation by a histidine-tryptophan switch. Journal of Biological Chemistry 278(29):27138-27143.
2. Farnegardh M et al. (2003) The three-dimensional structure of the liver X receptor beta reveals a flexible ligand-binding pocket that can accommodate fundamentally different ligands. Journal of Biological Chemistry 278(40):3881-3888.
3. Svensson S et al. (2003) Crystal structure of the heterodimeric complex of LXRalpha and RXRbeta ligand-binding domains in a fully agonistic conformation. EMBO Journal 22(18):4625-4633
4. Molteni et al. (2007) Compounds and compositions as LXR modulators. Published specification of application for patent for invention of the People's Republic of China, Application no. 200580004674.8
5. Liao Shu-zong et al. (2005) liver X receptor agonist. Published Specification of Application for Patent for Invention of the People's Republic of China, Application No. 02813426.5
6. Liao Shu-zong et al. (2004) steroid derivatives. Published Specification of Application for Patent for Invention of the People's Republic of China, Application No. 02807008.9
7. Liao Shu-zong et al. (2002) steroid derivatives. Published Specification of Application for Patent for Invention of the People's Republic of China, Application No. 00806958.1
8. Huang T H et al. (2005) A novel LXR-a activator identified from the natural product Gynostemma pentaphyllum. Biochemical Pharmacology 70:1298-1308.
9. Lee P S et al. (2006) Physicochemical characteristics and bioavailability of a novel intestinal metabolite of ginseng saponin (IH901) complexed with beta-cyclodextrin. International Journal of Pharmaceuticals 316:29-36
10. Cai B X et al. (2009) Ginsenoside Rb1 Suppresses Ultraviolet Radiation-Induced Apoptosis by Inducing DNA Repair. Biol. Pharm. Bull. 32(5):837-841
11. Bennett J et al. LXR agonists for the treatment of atherosclerosis: recent highlights. Annual Reports in Medicinal Chemistry Volume 43, Chapter 7, 103-117
12. Peng D et al. (2008) Antiatherosclerotic effects of a novel synthetic tissue-selective steroidal liver X receptor agonist in low-density lipoprotein receptor-deficient mice. Journal of Pharmacol. Exp. Ther. 327(2):332-342
13. Wojcicka G et al. (2007) Liver X receptors (LXRs). Part I: structure, function, regulation of activity, and role in lipid metabolism. Postepy Hig Med Dosw 61:736-759
14. Jamroz-Wisniewska A et al. (2007) Liver X receptors (LXRs). Part II: non-lipid effects, role in pathology, and therapeutic implications. Postepy Hig Med Dosw 61:760-785
15. Zelcer N et al. (2006) Liver X receptors as integrators of metabolic and inflammatory signaling. Journal of Clinical Investigation 116(3): 607-614.
16. Ai Zheng-lin et al. (2005) Research Advance in liver X receptors. World Chinese Journal of Digestology 13(16):2013-2015
17. Zheng Hui et al. (2001) Chemical Synthesis of novel LXRs ligands-epoxy cholanic acid methyl ester derivatives. Academiae Medicinae Militaris Tertiae 23(9):1091-1093
18. Zheng Hui et al. (2001) The Design and Synthesis of the Novel Selective LXRs' Ligand subtype. Journal of Chongqing Normal University (Natural Science Edition) 18(3):19-21
19. Song C et al. (2000) Cholestenoic acid is a naturally occurring ligand for liver X receptor alpha. Endocrinology 141(11):4180-4184
20. Song C et al. (2000) Selective activation of liver X receptor alpha by 6alpha-hydroxy bile acids and analogs. Steroids 65:423-427
21. Schultz J R et al. (2000) Role of LXRs in control of lipogenesis. Genes & Development 14:2831-2838
22. Spencer T A et al. (2001) Pharmacophore analysis of the nuclear oxysterol receptor LXRalpha. Journal of Medicinal Chemistry 44:886-897
23. Lehmann J M et al. (1997) Activation of the nuclear receptor LXR by oxysterols define a new hormone response pathway. Journal of Biological Chemistry 272(6):3137-3140
24. Willy P J et al. (1995) LXR, a nuclear receptor that defines a distinct retinoid response pathway. Genes & Development 9:1033-1045
25. Song C et al. (1994) Ubiquitous receptor: a receptor that modulates gene activation by retinoic acid and thyroid hormone receptors. Proc. Natl. Acad. Sci. U.S.A. 91 (23), 10809-10813
26. Shinar D M et al. (1994) NER, a new member of the gene family encoding the human steroid hormone nuclear receptor. Gene 147(2):273-276
27. Apfel R et al. (1994) A novel orphan receptor specific for a subset of thyroid hormone-responsive elements and its interaction with the retinoid/thyroid hormone receptor subfamily. Molecular and Cellular Biology 14(10):7025-7035

### DETAILS OF THE INVENTION

Nuclear receptor LXR is an important member of 49 nuclear receptors in human bodies and has irreplaceable regulatory effects on cholesterol and fat metabolisms. The regulation of the biological activities of LXR may have therapeutic effects on the conditions such as cardiovascular and cerebrovascular obstructions, non-insulin-dependent hyperglycemia, immune function disorders, central nerve functional degeneration, etc. We have developed a group of novel Chinese materia medica preparations aiming at nuclear receptor LXR by using a computer-simulated docking technology and a process for determining the biological activities of human cells cultured ex vivo. The group of preparations are features by simple ingredients, low costs, easy to prepare, etc. No significant adverse effects have been found in the initial acute toxicology analysis carried out by using mice.

### EXAMPLES

### Example 1

An edible liquor, Beijing Erguotou Liquor (alcohol content: 56%) 2500 millilitres, was used, and 100 grams sodium hydroxide solid (pharmaceutical-grade) was added therein and stirred to dissolve, and 100 grams of a total gypenosides extract (total gypenosides content>98%) (Xi'an Acetar Bio-Tech Co., Ltd.) was heated to dissolve. After the solution was cooled to room temperature, 300 millilitres of a 30% aqueous hydrogen peroxide solution were slowly added dropwise, with the solution standing at room temperature for 24 hours, then a white precipitate was filtered off. The supernatant was collected, then 100 millilitres of a 30% aqueous hydrogen peroxide solution were added dropwise, with the solution standing at room temperature for 48 hours and filtered, the supernatant was collected. The supernatant was neutralized with 2N diluted hydrochloric acid to pH 7, and ethanol was removed by distilling at 40-50° C. under reduced pressure. An oily solid appeared in the solution. Insoluble matter was collected, dried, and pulverized, and then a solid was collected. 64 g of derivatives of total gypenosides was obtained.

### Example 2

Hydroxypropyl-beta cyclodextrin was heated and dissolved to obtain a 40% aqueous solution, the total gypenoside derivatives were added in proportion (cyclodextrin:total gypenoside derivatives=4:1), heated for dissolution, and heated to concentrated the same, and dried by suction at 70° C. to remove water content; the solid obtained was pulverized to obtain a cyclodextrin inclusion compound. It was able to prepare oral dosage forms such as oral liquid, hard capsule, tablet, etc. by using the inclusion compound. It was also able to prepare intravenous injection solution by using the inclusion compound. The products was able to upregulate the biological activities of the LXR receptor.

### Example 3

40 millilitres of deionized water was heated to 70° C., 250 milligrams of Carbomer 940 was added, and stirred to dissolve. 250 milligrams of total gypenoside derivatives were dissolved in 3 millilitres of anhydrous ethanol and 1 millilitre of glycerol solution, and then they were added to the above solution. 2 millilitres of propylene glycol, 0.5 millilitres of Tween 80, 0.5 gram of stearic acid, 100 milligrams of vitamin B3, 60 milligrams of vitamin B6, 120 milligrams of arbutin, 360 milligrams of L-vitamin C, 30 milligrams of coenzyme Q, 0.3 millilitres of water-soluble azone, and water-soluble combined antibacterial agents were added in succession into the above solution at 70° C. under stirring, and triethanolamine was added dropwise to produce a neutral pH value. A few drops of rosemary oil were added dropwise, and a water-soluble skin care gel was obtained after being cooled. The skin care gel was able to promote the lipids metabolism of skin and anti-wrinkling.

### Example 4

A computer-simulated molecular docking method (Autodock 4.0 software, Scripps Institute, California, USA) was used to carry out the primary research on the interactions between the ginsenoside and the analogues thereof and nuclear receptor LXR (the crystal structure of LXRalpha's ligand-binding domain is 1UHL; the structure of LXRbeta is 1P8D, PDB database, USA). The bonding strength of some triterpenoid saponin compounds with LXR receptor was calculated by computer-simulated molecular docking method (the structures of the compounds were seen in the accompanying drawing of the specification):

| | | |
|---|---|---|
| Compounds | kI (LXRa) | kI (LXRb) |

| | | | | |
|---|---|---|---|---|
| 1 | 86.74 | nM | 26.35 | nM |
| 2 | 5.02 | nM | 32.88 | nM |
| 3 | 17.2 | nM | 160.6 | nM |
| 4 | 223.12 | nM | 3.16 | nM |
| 5 | 15.94 | nM | 1.06 | nM |
| 6 | 63.78 | nM | 1.94 | nM |
| 7 | 51.57 | nM | 46.52 | uM |
| 8 | 19.98 | nM | 4.60 | uM |
| 9 | 14.72 | nM | 2.6 | pM |
| 10 | 1.09 | nM | 15.56 | fM |
| 11 | 138.62 | nM | 12.3 | nM |
| 12 | 5.90 | nM | 110.58 | nM |
| 13 | 192.42 | nM | 113.56 | pM |

### Example 5

36 male C57BL/6 mice aged 12 weeks were equally divided into three experimental groups by body weights: a control group, a total gypenosides' group, and a derivatives' group. The feedstuff was high in lipids (21%) and high in cholesterol (2%); the drinking water was high in sugar (35% fruit pulp) (Huaqi hawthorn berry juice). The samples to be tested were added to the drinking water, and the dosage to be taken was 250 milligrams/per kilogram of body weight according to the amount of the drinking water. The feeding period of time was 21 days. The differences among the three experimental groups were shown in the following table. It can be presumed that the derivatives' group is more effective in activating LXR receptor as compared to the total gypenosides' group by data analysis.

TABLE 1
Physiological indexes of experimental mice

| | | food intake | water drinking |
|---|---|---|---|
| | body | amount g/per | amount g/per |
| | weight/g | mouse/per day | mouse/per day |
| control group | 19.8 ± 1.0 | 2.0 ± 0.4 | 5.4 ± 2.6 |
| total | 19.8 ± 2.0 | 2.1 ± 0.26 | 5.8 ± 2.0 |
| gypenosides' | | | |
| group | | | |
| derivatives' | 20.2 ± 1.2 | 2.1 ± 0.49 | 5.2 ± 1.7 |
| group | | | |

*T tests were carried out. The P values of the body weight, food intake amount, and water drinking amount of the total gypenosides' group were 0.489, 0.377, and 0.346, respectively. The P values of the body weight, food intake amount, and water drinking amount of the derivatives' group were 0.279, 0.135, and 0.431, respectively. Tail=1, type: double-sample variance hypothesis, in which the experiment data were all compared with the control group.

TABLE 2
Contents of total cholesterol and triglyceride
in serum and liver of experimental mice

| | total cholesterol | | triglyceride | |
|---|---|---|---|---|
| | | liver | | Liver |
| | serum | μmol/g | serum | μmol/g |
| | mmol/L | tissue | μmol/L | tissue |
| control group | 1.58 ± 0.17 | 5.94 ± 1.00 | 73.4 ± 17.9 | 4.75 ± 1.69 |
| total | 1.84 ± 0.32 | 5.15 ± 1.01 | 73.4 ± 13.9 | 8.73 ± 2.79 |
| gypenosides' | | | | |
| group | | | | |
| derivatives' | 1.66 ± 0.30 | 5.83 ± 1.36 | 81.6 ± 23.3 | 7.71 ± 2.00 |
| group | | | | |

*T tests were carried out. The P values of the contents of total cholesterol and triglyceride in sera of the mice of the total gypenosides' group were 0.0133 and 0.498, respectively; and the P values of the contents of total cholesterol and triglyceride in livers of the mice of the same group were 0.0397 and 0.000266, respectively. The P values of the contents of total cholesterol and triglyceride in sera of the derivatives' group were 0.238 and 0.482, respectively; and the P values of the contents of total cholesterol and triglyceride in livers of the mice of the same group were 0.421 and 0.000541, respectively.

### Example 6

### Measurement of Contents of Reducing Groups in Total Gypenosides and Derivatives Thereof by Using an Anthraquinone Method

A 80% sulphuric acid solution was prepared, anthraquinone (Beijing Chemical Reagent Company) and sulfourea (Sinopharm Chemical Reagent Co., Ltd.) were added, with the final concentrations being 0.4% and 1.6%, respectively. The test liquid could be stored at room temperature for a short period of time. Glucose standard solution was precisely prepared by using a weighing bottle and serially diluted into standard solutions containing 0-4 milligrams per millilitre. 50 microlitres of 98% sulphuric acid were added to a 96-well ELISA plate, and 10 microlitres of the standard solution or sample solution were added, then 200 microlitres of a test solution was further added, and was kept standing at room temperature for more than 30 minutes, then absorbance was measured at 620 nanometres wavelength by using an ELISA Reader, and the contents of the reducing groups in total gypenosides and derivatives thereof were calculated according to a standard curve.

### Example 7

### Measurement of Contents of Sapogenin in Total Gypenosides and Derivatives Thereof by Using Perchloric Acid-Vanillin-Phosphoric Acid Method

A 70% phosphoric acid aqueous solution containing 0.1% vanillin was prepared as the test solution. Cholesterol or ginsenoside Rb1 standard was used as standard substance and was quantitatively prepared into standard solutions containing 0-10 milligrams per millilitre. 100 microlitres of the standard solution or solution to be tested was taken up and into which was added 0.2 microlitres of perchloric acid, water bathing at 60° C. was carried out for 10 minutes, 0.5 microlitres of a test solution was added, water bathing at 60° C. was carried out for 5 minutes. After being cooled to room temperature, 0.2 microlitres were taken into a 96-well ELISA plate, absorbance was measured at 530 or 620 nanometre wavelength by using an ELISA Reader, and the contents of sapogenin in total gypenosides and derivatives thereof were calculated according to a standard curve.

### Example 8

### Detection of Total Gypenosides and Derivatives Thereof Using Thin-Plate Chromatography

A pre-fabricated silica gel chromatographic plate (Qingdao Haiyang Chemical Co., Ltd.) of 25×75 millimetres was selected, the spreading solvent was a saturated aqueous solution of chloroform:ethyl acetate:methanol (15:40:20), the colour developing agent was anhydrous ethanol solution in 10% sulphuric acid, and the developing condition was hot air blowing by a blower.

### Example 9

### Biological Activity Assay Method of Nuclear Receptor LXR Carried Out by Using Human Cells Cultured Ex Vivo

A human embryonic kidney cell strain HEK293 was selected and cultured in 48-well cell culture plates in a cell culture incubator with 5% carbon dioxide at 37° C. by using DMEM and 10% fetal bovine serum. When the cells were spread over 20% of the culture surface, they were transfected with LXR and RXRalpha expression plasmid and report gene plasmid using calcium phosphate precipitation process. For each 48-well plate, 10-20 micrograms of report gene plasmid (LXRE-c-fos promoter-firefly luciferase, Promega pGL3 vector), 0.2 micrograms of internal control report gene (CMV promoter-Renilla luciferase, Promega vector), 2 micrograms of LXR expression plasmid (pSG5-hLXRalpha or rLXRbeta, Strategene vector), and 2 micrograms of RXRalpha expression plasmid (pSG5-hRXRalpha, Strategene vector) were transfected. The culture fluid (a serum-free culture medium can be used) was changed 6 hours after the transfection and recombinant surface growth factor was supplemented to maintain the cell growth status. Samples to be tested were added to the culture medium six hours later. If the samples to be tested were dissolved in ethanol, the final ethanol concentration was not higher than 0.2%. After twenty four to thirty six hours, the expression level of the report gene of the cells was detected by using a Dual Luciferase Assay Kit (Promega Corporation) and a luminescence instrument (Xian).

### Example 10

Oral acute toxicity test was carried out by using mice and the acute toxicity of total gypenoside derivatives was assayed. ICR mice (10 male and female mice each, body weight 19-22 g) were used in this experiment, and they were fed in a barrier environment (ventilation 15 times/per hour for the feeding chamber, room temperature of 22-24° C., humidity of 40-60%, and 12 hours of bright and dark artificial illumination). 0.39 g/mL of suspension of total gypenoside derivatives was fed by intragastric administration with a needle at 0.25 mL per kilogram of body weight, no animal died after 7 days of intragastric administration, no observable manifestation of intoxication was found either, and the lethal dose (LD₅₀) was greater than 9.61 gram per kilogram of body weight.

1. Use of ex-vivo cultured human cells in detecting the biological transcription activities of a nuclear receptor, liver X receptor (LXR), so as to determine pharmacodynamic effects of Chinese materia medica preparations.

2. A process for preparing derivatives of natural triterpenes extract with an aqueous solution comprising sodium hydroxide, ethanol, and hydrogen peroxide, so as to improve the pharmaceutical values of Chinese materia medica preparations.

3. The process of claim 2, wherein the content of sodium hydroxide in the aqueous solution is in the range of 1-20%, the content of ethanol is in the range of 10-80%, and the content of hydrogen peroxide is the range of 1-10%.

4. The process of claim 3, wherein the content of sodium hydroxide in the aqueous solution is 4%, the content of ethanol is 50%, and the content of hydrogen peroxide is 4%.

5. The process of claim 1, wherein the content of the triterpenes extract is consistent with the corresponding content of sodium hydroxide.

6. The process of claim 5, wherein the triterpenes compounds comprise Radix Ginseng extracts, Fiveleaf Gynostemma Herb extracts, Ganoderma extracts, Radix Astragali seu Hedysari extracts, and extracts of others vital energy-invigorating Chinese materia medica.

7. The process of claim 2, wherein the pharmaceutical values comprise the pharmacodynamic effects in regulating the biological activities of LXR, such as reducing the cholesterol accumulation on the inner walls of arteries, regulating the immune system, improving the central nervous system, and improving the conditions of non-insulin-dependent diabetes mellitus.
